(19) United States
(12) Patent Application Publication
(10) Pub. No.: US 2023/0056370 A1
(43) Pub. Date: Feb. 23, 2023

(54) CONSTRUCTION METHOD OF FINE-GRAINED INFECTIOUS DISEASE SIMULATION MODEL

(71) Applicant: BEIHANG UNIVERSITY, Beijing (CN)

(72) Inventors: Honghao SHI, Beijing (CN); Xin LIN, Beijing (CN); Jiawei JIANG, Beijing (CN); Lida GUO, Beijing (CN); Jingyuan WANG, Beijing (CN)

(21) Appl. No.: 17/488,164

(22) Filed: Sep. 28, 2021

Related U.S. Application Data
(63) Continuation of application No. PCT/CN2021/117650, filed on Sep. 10, 2021.

(30) Foreign Application Priority Data
Aug. 23, 2021 (CN) 2021109695788

(51) Int. Cl. G16H 50/80

(52) U.S. Cl. CPC G16H 50/80

(57) ABSTRACT

A construction method of a fine-grained infectious disease simulation model is disclosed. The construction method includes: obtaining a population movement flow between multiple target regions within a predetermined time period; dividing the predetermined time period into multiple time periods based on a time mode; dividing the multiple target regions into multiple spatial nodes based on a spatial mode; and constructing a simulation model according to the population movement flow, the multiple time periods, and the multiple spatial nodes. By the method, the dynamic modeling of the development of infectious diseases is completed; the sub model modeling under different time modes is completed; and the fine-grained modeling under different spatial modes is completed.

### CROSS REFERENCE TO RELATED APPLICATION

This patent application is a continuation application of PCT/CN2021/117650 having an international filing date of Sep. 10, 2021 which further claims the benefit of Chinese Patent Application No. 202110969578.8 filed on Aug. 23, 2021, the disclosure of which is incorporated by reference herein in its entirety as part of the present application.

### TECHNICAL FIELD

The present disclosure relates to the technical field of epidemiology, dynamic model and computer application, and more specifically, to a construction method of a fine-grained infectious disease simulation model.

### BACKGROUND ART

As of June 2021, Corona Virus Disease 2019 (COVID-19) has diagnosed more than 170 million people worldwide. In the spread process of COVID-19, urban spread is the most serious. In order to simulate the spread of the epidemic and facilitate defense, relevant researchers have set up epidemic prediction or simulation tools. However, the existing epidemic prediction or simulation tools are usually compartment models based on SEIR, which can not distinguish the different spread modes in city and village, and can not quantitatively analyze the major factors affecting the spread of the epidemic, such as traffic flow.

Therefore, how to constructing a model that can simulate the spread mode of the epidemic from multiple dimensions such as time and space has become a key problem in the current research.

### SUMMARY

In view of the above problems, the disclosure provides a construction method of a fine-grained infectious disease simulation model to solve at least some of the above technical problems. Through this method, the spread mode of the epidemic can be simulated from multiple dimensions such as time and space, so as to realize the fine-grained infectious disease prediction simulation.

The disclosure provides a construction method of a fine-grained infectious disease simulation model, including:

obtaining a population movement flow between multiple target regions within a predetermined time period;

dividing the predetermined time period into multiple time periods based on a time mode;

dividing the multiple target regions into multiple spatial nodes based on a spatial mode; and

constructing a simulation model according to the population movement flow, the multiple time periods, and the multiple spatial nodes.

Further, the obtaining a population movement flow between multiple target regions within a predetermined time period includes:

obtaining a case data, a population number and a spatial range data in the multiple target regions;

obtaining a population movement index between the multiple target regions according to the case data, the population number and the spatial range data; and

performing a data characterization on the population movement index to obtain the population movement flow between the multiple target regions.

Further, the performing a data characterization on the population movement index includes:

dividing each of the target regions according to a spatial scale of n meters× n meters and a time scale of s hours to obtain multiple intervals corresponding to all the target regions; and

integrating a signaling data of the users' mobile phones at a current time scale in each of the intervals, obtaining a movement flow of the users at the current time scale in each of the intervals, and realizing the data characterization on the population movement index.

Further, the dividing the predetermined time period into multiple time periods based on a time mode includes:

equidistantly dividing the predetermined time period; or,

dividing the predetermined time period according to a work and rest regular pattern of the population.

Further, the simulation model is represented as:

$$\frac{{dS}_{i}}{dt} = - {\sum }_{h = 1}^{3} ( {β}_{1 , h}^{i} \frac{{S}_{i}}{{N}_{i}} {\sum }_{j = l}^{l} \frac{{T}_{hjit} \cdot {C}_{j}}{{N}_{j}} {I}_{j} ) - {β}_{2}^{i} \frac{{S}_{i}}{{N}_{i}} {I}_{i} - {\sum }_{h = 1}^{3} ( q {β}_{1 , h}^{i} \frac{{S}_{i}}{{N}_{i}} {\sum }_{j = l}^{l} \frac{{T}_{hjit} \cdot {C}_{j}}{{N}_{j}} {p}_{j} ) - q {β}_{2} \frac{{S}_{i}}{{N}_{i}} {p}_{i} \qquad ( 1 )$$

$$\frac{{dE}_{i}}{dt} = {\sum }_{h = 1}^{3} ( {β}_{1 , h}^{i} \frac{{S}_{i}}{{N}_{i}} {\sum }_{j = l}^{l} \frac{{T}_{hjit} \cdot {C}_{j}}{{N}_{j}} {I}_{j} ) + {β}_{2}^{i} \frac{{S}_{i}}{{N}_{i}} {I}_{i} + {\sum }_{h = 1}^{3} ( q {β}_{1 , h}^{i} \frac{{S}_{i}}{{N}_{i}} {\sum }_{j = l}^{l} \frac{{T}_{hjit} \cdot {C}_{j}}{{N}_{j}} {p}_{j} ) + q {β}_{2} \frac{{S}_{i}}{{N}_{i}} {p}_{i} - {α}_{E} {E}_{i} \qquad ( 2 )$$

$$\frac{{dP}_{i}}{dt} = {α}_{E} {E}_{i} - {α}_{p} {P}_{i} \qquad ( 3 )$$

$$\frac{{dI}_{i}}{dt} = {α}_{p} {P}_{i} - γ {I}_{i} \qquad ( 4 )$$

$$\frac{{dR}_{i}}{dt} = γ {I}_{i} \qquad ( 5 )$$

wherein, Sᵢ represents susceptible persons at an i-th spatial node; Eᵢ represents exposed persons at the i-th spatial node; Pᵢ represents pre-symptom infected persons at the i-th spatial node; Iᵢ represents infected persons at the i-th spatial node; Rᵢ represents removers at the i-th spatial node; h represents a time mode; Tₕⱼᵢₜ represents a movement flow on day t from a j-th spatial node to the i-th spatial node in the h-th time mode; β represents infection rate parameters; α represents morbidity parameters; γ represents a removal rate parameter; Cⱼ represents a multiple of a total population in the j-th spatial node relative to a number of people holding the mobile phones; Nᵢ represents a total population number at the i-th spatial node; q represents a change ratio of an infection rate between pre-symptom infected persons and post-symptom infected persons.

Compared with the prior art, the construction method of a fine-grained infectious disease simulation model recorded in the disclosure has the following beneficial effects:

the dynamic modeling of the development of infectious diseases is completed;

the sub model modeling under different time modes is completed; and

the fine-grained modeling under different spatial modes is completed.

Other features and advantages of the disclosure will be described in the following description, and will become apparent in part from the description, or will be understood by implementing the disclosure. The object and other advantages of the disclosure can be realized and obtained by the structure specially pointed out in the description, claims and drawings.

The technical scheme of the disclosure is further described in detail below through the accompanying drawings and embodiments.

### BRIEF DESCRIPTION OF THE DRAWINGS

The accompanying drawings are used to provide a further understanding of the disclosure and form part of the description. They are used to explain the disclosure together with the embodiments of the disclosure and do not constitute a limitation of the disclosure. In the drawings:

FIG. 1 is the flowchart of the construction method of a fine-grained infectious disease simulation model provided by the embodiment of the present disclosure.

FIG. 2 is the simulation model diagram provided by the embodiment of the present disclosure.

### DETAILED DESCRIPTION OF THE EMBODIMENTS

Exemplary embodiments of the present disclosure will be described in more detail below with reference to the accompanying drawings. Although exemplary embodiments of the present disclosure are shown in the accompanying drawings, it should be understood that the present disclosure may be implemented in various forms and should not be limited by the embodiments set forth herein. On the contrary, these embodiments are provided to enable a more thorough understanding of the present disclosure and to fully convey the scope of the present disclosure to those skilled in the art.

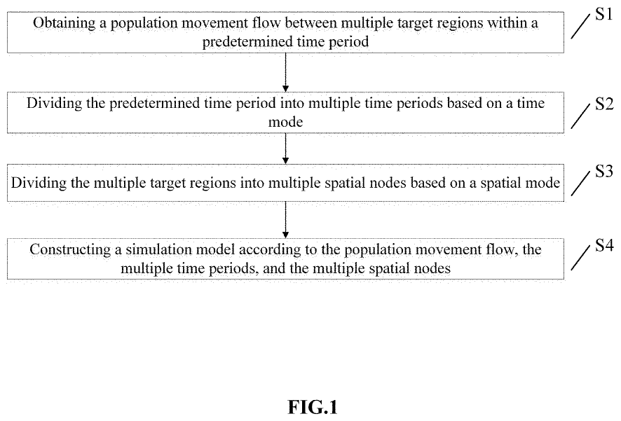

As shown in FIG. 1, the disclosure provides a construction method of a fine-grained infectious disease simulation model, including:

obtaining a population movement flow between multiple target regions within a predetermined time period;

dividing the predetermined time period into multiple time periods based on a time mode;

dividing the multiple target regions into multiple spatial nodes based on a spatial mode; and

constructing a simulation model according to the population movement flow, the multiple time periods, and the multiple spatial nodes.

Through this method, the spread mode of the epidemic can be simulated from multiple dimensions such as time and space, so as to realize fine-grained infectious disease prediction simulation.

The content of the above method will be described in detail below.

In the above method, the obtaining a population movement flow between multiple target regions within a predetermined time period includes:

obtaining a case data, a population number and a spatial range data in the multiple target regions; in the present embodiment, the case data (number of confirmed cases, number of cured cases, number of deaths, etc.) of each sub district office, the population of each sub district office and the spatial range (longitude and latitude) of each sub district office in Wuhan from January 2020 to March 2020 were collected;

obtaining a population movement index between the multiple target regions according to the case data, the population number and the spatial range data;

performing a data characterization on the population movement index to obtain the population movement flow between the multiple target regions; the data characterization (or data embedding) refers to the transformation of multi-source and heterogeneous raw data into vectors that can be directly applied by the model through a series of algorithms; for example, “Jan. 13, 2020” and “20.01.15” expressed in natural language are characterized as “18274” and “18276” (the days difference from 1970.1.1); during the epidemic period, the signaling data of users' mobile phones recorded the mobile phone access track of the users of each cellular base station; therefore, in this embodiment, the population movement flow of each sub district office in Wuhan was mainly measured by the mobile phone signaling data, that is, the signaling data of the users' mobile phones was integrated into the travel flow of mobile phone users; the specific was dividing each of the target regions according to a spatial scale of n meters× n meters and a time scale of s hours to obtain multiple intervals corresponding to all the target regions; in this embodiment, it was divided specifically according to the spatial scale of 500 m×500 m and the time scale of 1 hour; then, the grid flow data in each of the intervals were integrated to form user travel flow; that is, the users' mobile phone signaling data under the current time scale in each of the intervals was integrated to obtain the users' movement flow under the current time scale in each of the intervals, and realize the data characterization of the population movement index.

In the above method, the dividing the predetermined time period into multiple time periods based on a time mode includes: equidistantly dividing the predetermined time period, or, dividing the predetermined time period according to a work and rest regular pattern of the population. In the embodiment, the predetermined time period was divided according to the work and rest regular pattern of the population, which was specifically divided into 7:00-9:00, 16:00-18:00 and other time periods; and 7:00-9:00 were the morning rush hours and 16: 00-18:00 were the evening rush hours.

In the above method, the multiple target regions are divided into multiple spatial nodes based on a spatial mode. In the embodiment, taking Wuhan as an example, 161 sub district offices were divided into 99 spatial nodes. On this basis, the development process of infectious diseases within each node, that is, between nodes, was modeled.

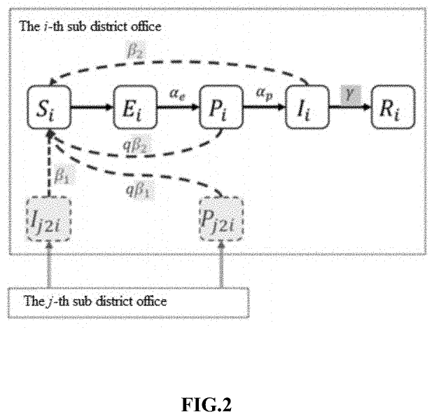

In the above method, a simulation model is constructed according to the population movement flow, the multiple time periods, and the multiple spatial nodes. Specifically, as shown in FIG. 2, the simulation model can be expressed as:

$$\frac{{dS}_{i}}{dt} = - {\sum }_{h = 1}^{3} ( {β}_{1 , h}^{i} \frac{{S}_{i}}{{N}_{i}} {\sum }_{j = l}^{l} \frac{{T}_{hjit} \cdot {C}_{j}}{{N}_{j}} {I}_{j} ) - {β}_{2}^{i} \frac{{S}_{i}}{{N}_{i}} {I}_{i} - {\sum }_{h = 1}^{3} ( q {β}_{1 , h}^{i} \frac{{S}_{i}}{{N}_{i}} {\sum }_{j = l}^{l} \frac{{T}_{hjit} \cdot {C}_{j}}{{N}_{j}} {p}_{j} ) - q {β}_{2} \frac{{S}_{i}}{{N}_{i}} {p}_{i} \qquad ( 1 )$$

$$\frac{{dE}_{i}}{dt} = {\sum }_{h = 1}^{3} ( {β}_{1 , h}^{i} \frac{{S}_{i}}{{N}_{i}} {\sum }_{j = l}^{l} \frac{{T}_{hjit} \cdot {C}_{j}}{{N}_{j}} {I}_{j} ) + {β}_{2}^{i} \frac{{S}_{i}}{{N}_{i}} {I}_{i} + {\sum }_{h = 1}^{3} ( q {β}_{1 , h}^{i} \frac{{S}_{i}}{{N}_{i}} {\sum }_{j = l}^{l} \frac{{T}_{hjit} \cdot {C}_{j}}{{N}_{j}} {p}_{j} ) + q {β}_{2} \frac{{S}_{i}}{{N}_{i}} {p}_{i} - {α}_{E} {E}_{i} \qquad ( 2 )$$

$$\frac{{dP}_{i}}{dt} = {α}_{E} {E}_{i} - {α}_{p} {P}_{i} \qquad ( 3 )$$

$$\frac{{dI}_{i}}{dt} = {α}_{p} {P}_{i} - γ {I}_{i} \qquad ( 4 )$$

$$\frac{{dR}_{i}}{dt} = γ {I}_{i} \qquad ( 5 )$$

wherein, Sᵢ represents susceptible persons at an i-th spatial node; Eᵢ represents exposed persons at the i-th spatial node; Pᵢ represents pre-symptom infected persons at the i-th spatial node; Iᵢ represents infected persons at the i-th spatial node; Rᵢ represents removers at the i-th spatial node; h represents a time mode; Tₕⱼᵢₜ represents a movement flow on day t from a j-th spatial node to the i-th spatial node in the h-th time mode; β represents infection rate parameters; α represents morbidity parameters; γ represents a removal rate parameter; Cⱼ represents a multiple of a total population in the j-th spatial node relative to a number of people holding the mobile phones; Nᵢ represents a total population number at the i-th spatial node; q represents a change ratio of an infection rate between pre-symptom infected persons and post-symptom infected persons. For example, if the infection rate of the pre-symptom infected persons is 100, then the infection rate of the pre-symptom infected persons is 100*q.

The embodiment of the disclosure provides a construction method of a fine-grained infectious disease simulation model, which transforms the application mode of SEIR and other compartment models and realizes a dynamic parameter mechanism based on crowd activity intensity (traffic flow). The dynamic model built by this method can review the outbreak of COVID-19 in Wuhan in 2020, and analyze, compare, and summary the characteristics of the spread of the epidemic under different time models (morning rush hour, evening rush hour, and others) and different spatial models (provinces, municipalities, districts and sub district office). On this basis, the prediction and simulation of infectious diseases can be realized.

Obviously, those skilled in the art can make various modifications and variations to the disclosure without departing from the spirit and scope of the disclosure. Thus, if these modifications and variations of the disclosure fall within the scope of the claims of the disclosure and its equivalent technology, the disclosure is also intended to include these modifications and variations.

What is claimed is:

1. A construction method of fine-grained infectious disease simulation model, comprising:
obtaining a population movement flow between a plurality of target regions within a predetermined time period;
dividing the predetermined time period into a plurality of time periods based on a time mode;
dividing the plurality of target regions into a plurality of spatial nodes based on a spatial mode; and
constructing a simulation model according to the population movement flow, the plurality of time periods, and the plurality of spatial nodes.

2. The construction method of fine-grained infectious disease simulation model of claim 1, wherein the obtaining a population movement flow between a plurality of target regions within a predetermined time period comprises:
obtaining a case data, a population number and a spatial range data in the plurality of target regions;
obtaining a population movement index between the plurality of target regions according to the case data, the population number and the spatial range data; and
performing a data characterization on the population movement index to obtain the population movement flow between the plurality of target regions.

3. The construction method of fine-grained infectious disease simulation model of claim 2, wherein the performing a data characterization on the population movement index comprises:
dividing each of the target regions according to a spatial scale of n meters× n meters and a time scale of s hours to obtain a plurality of intervals corresponding to all the target regions; and
integrating a signaling data of the users' mobile phones at a current time scale in each of the intervals, obtaining a movement flow of the users at the current time scale in each of the intervals, and realizing the data characterization on the population movement index.

4. The construction method of fine-grained infectious disease simulation model of claim 1, wherein the dividing the predetermined time period into a plurality of time periods comprises:
equidistantly dividing the predetermined time period; or,
dividing the predetermined time period according to a work and rest regular pattern of the population.

5. The construction method of fine-grained infectious disease simulation model of claim 1, wherein the simulation model is represented as:
$$\frac{{dS}_{i}}{dt} = - {\sum }_{h = 1}^{3} ( {β}_{1 , h}^{i} \frac{{S}_{i}}{{N}_{i}} {\sum }_{j = l}^{l} \frac{{T}_{hjit} \cdot {C}_{j}}{{N}_{j}} {I}_{j} ) - {β}_{2}^{i} \frac{{S}_{i}}{{N}_{i}} {I}_{i} - {\sum }_{h = 1}^{3} ( q {β}_{1 , h}^{i} \frac{{S}_{i}}{{N}_{i}} {\sum }_{j = l}^{l} \frac{{T}_{hjit} \cdot {C}_{j}}{{N}_{j}} {p}_{j} ) - q {β}_{2} \frac{{S}_{i}}{{N}_{i}} {p}_{i} \qquad ( 1 )$$
$$\frac{{dE}_{i}}{dt} = {\sum }_{h = 1}^{3} ( {β}_{1 , h}^{i} \frac{{S}_{i}}{{N}_{i}} {\sum }_{j = l}^{l} \frac{{T}_{hjit} \cdot {C}_{j}}{{N}_{j}} {I}_{j} ) + {β}_{2}^{i} \frac{{S}_{i}}{{N}_{i}} {I}_{i} + {\sum }_{h = 1}^{3} ( q {β}_{1 , h}^{i} \frac{{S}_{i}}{{N}_{i}} {\sum }_{j = l}^{l} \frac{{T}_{hjit} \cdot {C}_{j}}{{N}_{j}} {p}_{j} ) + q {β}_{2} \frac{{S}_{i}}{{N}_{i}} {p}_{i} - {α}_{E} {E}_{i} \qquad ( 2 )$$
$$\frac{{dP}_{i}}{dt} = {α}_{E} {E}_{i} - {α}_{p} {P}_{i} \qquad ( 3 )$$
$$\begin{matrix}\frac{{dI}_{i}}{dt} = {α}_{p} {P}_{i} - γ {I}_{i} & ( 4 )\end{matrix}$$
$$\frac{{dR}_{i}}{dt} = γ {I}_{i} \qquad ( 5 )$$
wherein, Sᵢ Eᵢ represents exposed persons at the i-th spatial node; Pᵢ
Iᵢ represents infected persons at the i-th spatial node; Rᵢ
Tₕⱼᵢₜ represents a population movement flow on day t from a j-th spatial node to the i-th spatial node in the h-th time mode; β represents infection rate parameters; α represents morbidity parameters; γ represents a removal rate parameter; Cⱼ represents a multiple of a total population in the j-th spatial node relative to a number of people holding the mobile phones; Nᵢ represents a total population number at the i-th spatial node; and q represents a change ratio of an infection rate between pre-symptom infected persons and post-symptom infected persons.
